(19) United States
(12) Patent Application Publication
(10) Pub. No.: US 2016/0346529 A1
(43) Pub. Date: Dec. 1, 2016

(54) ELECTRIC MASK COMPRISING AT LEAST TWO COMPARTMENTS

(71) Applicant: L'OREAL, Paris (FR)

(72) Inventors: Jennyfer CAZARES DELGADILLO, Paris (FR); Thi Hong Lien PLANARD-LUONG, Bures sur Yvette (FR)

(21) Appl. No.: 15/116,520

(22) PCT Filed: Jan. 30, 2015
(86) PCT No.: PCT/FR2015/050224

§ 371 Date: Aug. 4, 2016

(30) Foreign Application Priority Data
Feb. 4, 2014 (FR) 1450841
Jul. 29, 2014 (FR) 1457331

(51) Int. Cl. A61N 1/04; A61N 1/32; A45D 44/00; A61M 35/00

(52) U.S. Cl. CPC A61N 1/0448; A61M 35/00; A61N 1/325; A45D 44/002; A61M 2210/0606

(57) ABSTRACT

The present application relates to an article for the cosmetic treatment of a body area by an electric current, comprising:

(i) a support (13) including:
a first compartment (3) comprising a first cosmetic composition and a first electrode (31),
a second compartment (6) comprising a second cosmetic composition and a second electrode (61),

(ii) at least one counter electrode (14, 32, 62).

The present invention relates to articles for the cosmetic treatment of human keratin materials with an electric current.

Within the context of the present invention, an “article” is understood to mean a mask, a patch, a pad, a strip or a bandage capable of being applied to human keratin materials.

The expression “cosmetic product” is understood to mean any composition as defined in Council Directive 93/35/EEC of 14 Jun. 1993.

Passive masks for application of cosmetic composition are known. U.S. Pat. No. 6,702,792, for example, describes a cellulose mask impregnated with a facial lotion.

However, the amount of molecules administered via the passive route remains low. In order to improve this aspect, active patches or masks have been developed that use electrodes connected to a power source.

It is thus known to treat human keratin materials using iontophoresis devices (J. Singh, K. S. Bhatia, Topical iontophoretic drug delivery: pathways, principles, factors and skin irritation, Med. Res. Rev., vol. 16, no. 3, 285-296, 1996).

Iontophoresis allows the diffusion of active agents through the skin by virtue of electrical stimulation in a non-invasive manner The current applied may be adjustable in terms of intensity and direction (anodal or cathodal). The transcutaneous diffusion of the molecules via iontophoresis is based on two principles, namely electrorepulsion and electroosmosis.

Electrorepulsion is the migration of an ionized molecule by repulsion of charges of the same sign. Thus, if a substance has a positive charge, it will diffuse through the skin at the anode (+).

Electroosmosis is the migration of a molecule, even a non-ionized molecule, by entrainment associated with the flow of water from the anode to the cathode during iontophoresis. The migration is due in particular to the negative charge of the skin. Under the effect of a current, the water or a solvent entrains dissolved substances as it migrates.

Patents U.S. Pat. No. 6,157,858, U.S. Pat. No. 7,069,088, U.S. Pat. No. 6,157,858 or U.S. Pat. No. 7,069,088 describe articles comprising at least one pair of electrodes. The electrodes are placed on a malleable support. They are powered by a generator external to the support located within a housing. Before placing the article on the chosen body area, a cosmetic composition is applied to this area. The article is then placed on the face and the electrodes are powered by electric current. The treatment time varies between 15 minutes and 1 hour.

Such an article allows only one cosmetic to be applied to the selected body area. Thus, the entirety of the area is treated with the same composition. Such a blanket treatment may cause problems in the case where the area includes regions of different natures. For example, if the selected area is the face, certain regions may be more oily than others. Some regions of the face may be more sensitive than others. Yet others may be more marked by deep or first wrinkles.

Furthermore, the composition is applied by the user to the target area before treatment, this possibly leading to a nonuniform distribution of the active principle.

CN 200951261 describes an article, the active electrode of which covers the whole of the face while the return electrode is attached to another part of the body. Electrical contacts and electric wires connect the electrodes to the power source. The latter is contained in an external housing. The mask is composed of an absorbent layer in contact with the skin, topped with a conductive layer made of rubber. The cosmetic composition is contained in the absorbent fabric.

The article described in patent U.S. Pat. No. 5,443,441 has a base structure similar to that described in CN 200951261, with the difference that the cosmetic composition is not contained in an absorbent fabric but in a reservoir separated from the skin by a semipermeable membrane.

Thus, the cosmetic composition is ready to use in the article. The user saves time since he has no need to apply the composition to his face. However, this article is not easy to manipulate because the power source is integrated into an external housing.

Furthermore, the active electrode may produce a nonuniform contact between the article and the body area and, therefore, a nonuniform distribution of the current over the skin.

However, a nonuniform distribution of the current may decrease the comfort of the user, for example by creating a prickling sensation or a sensation of burning locally, in the places where the current density is highest.

Lastly, these articles deliver the same cosmetic composition over the entirety of the selected body area. The entirety of this area is treated uniformly, without taking into account possible specificities.

There is therefore a need to provide an electrical article allowing various regions of a selected body area to be selectively and simultaneously treated. This article must be easy to manipulate.

There is also a need to provide an article allowing at least two different cosmetic compositions to be applied to two contiguous regions of a body area of a user.

There is also a need to decrease the bulk of existing articles.

There is additionally a need to propose an article that enables the uniform application of a cosmetic composition containing an active principle to each region of the selected area.

The invention aims to resolve all or some of the aforementioned needs and also to further improve the articles for cosmetic treatment of human keratin materials with an electric current.

According to a first of its aspects, a subject of the invention is an article for cosmetic treatment of a body area by an electric current, comprising:

(i) a support including:
a first compartment comprising a first cosmetic composition and a first electrode,
a second compartment comprising a second cosmetic composition and a second electrode,

(ii) at least one counter electrode.

In particular, the article according to the invention is a mask for the face.

The article according to the invention may in particular be used with a housing separate from the article, this housing comprising a power supply source. In this case, a new article is connected to the housing at the time of each treatment. The article is generally disposable after one use. The article operates in combination with the housing.

The article may also have its power supply source built in. It then operates autonomously. At the end of each treatment, the user throws away the article with its power supply source.

If the article is a mask, it may have openings for the eyes, the nose and/or the mouth.

The article may be occlusive or non-occlusive, for example being impermeable to steam.

The term “face” should be understood to mean the external area of the anterior part of a human head. The face comprises in particular the chin, the mouth, the lips, the philtrum, the nose, the cheeks, the cheekbones, the eyes, the eyebrows and the forehead.

Advantageously, each compartment covers one area selected from the chin, the lips, the nose, the cheeks, the cheekbones, the eyebrows, and the forehead, when the article is placed on the face.

According to the invention, an “electrode” is understood to be a positively charged electrode (anode) or a negatively charged electrode (cathode). This electrode is generally disposed on the external surface of the article so as to come into direct contact with the keratin materials. However, the electrode may also be inserted into the external wall of the article. In this case, it does not come into direct contact with the keratin materials. In general, the electrode is in contact with the area to be treated.

Throughout the text, the term “electrode” means a single insulated electrode. An electrode may be in the form of a ball or stud, for example. A “counter electrode” is understood to be a negatively charged electrode (cathode) or a positively charged electrode (anode). The charge of the counter electrode is opposite to that of the electrode.

Advantageously, the article comprises a first counter electrode and a second counter electrode. In particular, the first counter electrode is placed facing the first electrode and the second counter electrode is placed facing the second electrode. Thus, the user does not have to hold one or more counter electrodes in his hand. The article is easier to use.

The article may alternatively include a single counter electrode, especially if all the electrodes have the same polarity. The counter electrode is intended to come into contact with an area of the body of the person undergoing the care treatment. For example, it may be held between the person's fingers. In one configuration, the counter electrode may be placed on the border of the article, set back from the compartments. If this is the case, it is separated from the electrodes by an insulating space.

A “power supply system” is understood to be an electrical assembly that is able to induce a potential difference between the electrodes and the counter electrode. If the article is placed on the face and if the counter electrode is held in a hand, the potential difference makes it possible to establish a current between the face and the hand.

A “compartment” is understood to be a division made in an article in order to house cosmetic compositions while keeping them separate.

Advantageously, the first compartment and the second compartment are separated by a separation zone. The separation zone is generally devoid of cosmetic composition. Thus, the two compartments are easily distinguishable by the user. It is easy to place them facing a selected area.

### Electric Power Source

The article may comprise, within it, an electric power source.

The incorporation of the electric power source of the mask may make it possible to do away with electric wires connecting the mask to an external source. The ease of use of the mask is then increased and it becomes easier for the user to use the mask while moving about or in the bath.

Advantageously, the article comprises an electric power source, located on the support.

The electric power source may be attached to the article in a removable or non-removable manner.

The electric power source may, for example, be incorporated into the mask during its manufacture. The power source may be sandwiched between two outer layers of the article.

It is also possible for the electric power source to be installed in the article just before the treatment. It may then be offered to the user separately from the mask.

The electric power source may comprise a DC voltage source. As a variant, the electric power source may comprise an electronic circuit for varying the amplitude of the voltage generated over time. This electronic circuit may be a chopper, for example.

The electric power source may, for example, be connected to the electrodes without a cutout. In this case, the electrical resistance between the electrodes may be high enough so that the electric power source does not discharge when the article is not in operation.

It is also possible to start the electric power source by pushing on a portion of the mask in order to bring said electric power source into contact with a conductive track.

The electric power source may comprise a DC voltage source. As a variant, the electric power source may comprise an electronic circuit for varying the amplitude of the voltage generated over time. This electronic circuit may, for example, be a chopper or a constant current generator.

The electric power source may, for example, be connected to the electrodes without a cutout. In this case, the electrical resistance between the electrodes may be high enough so that the electric power source does not discharge when the article is not in operation.

It is also possible to start the electric power source by pushing on a portion of the mask in order to bring said electric power source into contact with a conductive track.

### Electrical Parameters

The electric power source may comprise any non-rechargeable battery or any accumulator. The potential difference between the electrodes is for example between 1.2 V and 24 V, preferably between 1.2 and 3.3 V. If appropriate, the passage of the current can create spot heating.

At an equivalent current density, the article can in particular deliver a current density, at the skin, of preferably less than or equal to 0.500 mA/cm², for example between 0.01 mA/cm² and 0.500 mA/cm², for example between 0.01 mA/cm² and 0.10 mA/cm².

### The Various Types of Currents

It is possible to use a direct current, an alternating current or a pulsating current to power the article according to the invention.

Preferably, the article is powered by a sequential current the basic element of which is a DC current, an AC current or a pulsed current.

The sequential current is obtained by installing a switch. A current switching device, in other words a switch, is installed with the generator. This switch makes it possible to change the active state of the current sent to each compartment. Specifically, the current is sent in sequence to each compartment. Each sequence lasts between 1 second and 1 minute, preferably between 1 second and 10 seconds.

The article may also be powered by a sequential current and a continuous current of low intensity.

Advantageously, the generator is designed so that the user can change the polarity of the current.

Thus, the article enables, at will, extraction of impurities from the body area, care of the body area or making up of the body area.

### Electrodes

The electrodes may have a visible free surface allowing them to come into direct contact with the skin.

The electrode may be flat, for example in the form of a flat disc or polygon.

The electrode may be porous. The electrodes may have various shapes and for example a surface intended to come into contact with the skin which is outwardly convex, outwardly concave, or flat. Preferably, the electrodes are smooth so as not to hurt the skin.

The electrodes may be formed by two spheres or rollers, which may or may not be able to rotate in respective housings.

The electrode may be hollow, being formed for example by stamping or bending an electrically conductive metal sheet.

Materials Able to Be Used to Produce the Electrodes

The material(s) forming the electrodes may be identical or different.

At least one electrode may comprise, for example:

a metal (chromium, stainless steel), for example

a noble metal (gold, titanium) which is inert with respect to the composition,

a metal plated with a noble metal,

an alloy,

a composite material (plastics material filled with carbon microfibers),

a conductive woven fabric,

a conductive nonwoven fabric,

a polymer material rendered conductive,

a fibrous material.

conductive polymeric fibers, for example as described in the publication CN101532190,

carbon fibers, for example as described in the publication JP2009179915,

silicones rendered conductive by the addition of conductive fillers such as silver, copper or carbon. Such silicones are supplied, for example, by the companies Saint Gobain, Plastics Performance and Aquitaine Caoutchouc 2000,

conductive metallic fabrics, supplied for example by the companies Utexbel and Cousin Biotech,

carbon-filled vinyl, supplied for example by the companies Copema and Rexam,

electrosurgical plates, supplied for example by the companies Copema and 3M,

intrinsically conducting polymers, supplied for example by the company Paniplast.

The article may include an electrically insulating layer, securely fastening the first and second electrodes. This layer may be an exterior layer, opposite the skin.

The “active area of an electrode” is understood to mean the area of an electrode in contact with the body area, when the article is in place on said body area.

The “active area of a compartment” is understood to mean the area of the compartment in contact with the body area, when the article is in place on said body area.

Advantageously, the active area of the first electrode is greater than one third of the active area of the first compartment.

The effectiveness of the treatment is optimized level with the first compartment. The treatment is more uniform over the body area located facing the first compartment.

Advantageously, the active area of the second electrode is greater than one third of the active area of the second compartment.

The effectiveness of the treatment is optimized level with the second compartment. The treatment is more uniform over the body area located facing the second compartment.

### Electrically Insulating Zone

The electrically insulating zone forms a separation zone between the compartments. It completely separates the compartments so as to electrically insulate them from one another. The electrically insulating zone provides electrical insulation.

The electrically insulating zone may have a visible free surface allowing them to come into direct contact with the skin.

The expression “ electrically insulating zone” is understood to mean a zone including an electrical insulator, also called a dielectric material. This zone prevents electrical current from passing between two compartments. It does not conduct electricity.

Advantageously, the compartments are separated by an electrically insulating zone.

More advantageously, the electrically insulating zone is designed to prevent the diffusion of a cosmetic composition between the compartments.

### Parameters of the Electrically Insulating Zone

The electrically insulating zone has an electrical conductivity of zero or almost zero and an infinite resistance (∞Ω).

The electrically insulating zone is also defined by its permittivity and by electric strength.

Advantageously, the electrically insulating zone has an electrical conductivity of less than 10⁻⁶ S.m⁻¹, preferably of less than 10⁻¹² S.m⁻¹.

Permittivity

The permittivity or dielectric constant of an insulator is expressed relative to that of air (equal to that of vacuum). It is represented by the letter epsilon ε and expressed in picofarads/meter.

The vacuum permittivity is equal to:

ε₀8,854187·10⁻¹²F·m⁻¹

The absolute permittivity of a material is the product of its relative permittivity (see table below) multiplied by the vacuum permittivity according to the formula:

ε=ε₀×ε_{R}

For Teflon it is 18.6 pF/m.

Permittivity and Dielectric Strength of Several Insulators

These values are approximate and may vary markedly as a function of the frequency, of the temperature, of the hygrometry or even of the atmospheric pressure.

Permittivity is also referred to as dielectric constant (symbol ∈ᵣ).

The dielectric strength is in kV/mm

| | | Dielectric strength |
|---|---|---|
| Insulator | Relative permittivity ∈ᵣ | (kV/mm) |
| Dry air | 1 | 4 |
| Rubber | 4 | 15 |
| Silicone rubber | 4.2 | — |
| Cardboard | 4 | 10 |
| Kapton ® | | 110 |
| Mica | 6 | 70 |
| Paper | 2 | 6 |
| Paraffin | 2.2 | — |
| PVC | 5 | 20 |
| Plexiglas | 3.3 | — |
| Polyester | 3.3 | — |
| Polyethylene | 2.25 | 18 |
| Polypropylene | 2.2 | — |
| Polystyrene | 2.4 | 24 |
| Polycarbonate | 2.9 | 30 |
| Steatite | 5.8 | — |
| Styroflex | 2.5 | — |
| Teflon | 2.1 | 17 |
| Glass | 5 to 7 | 10 |
| Glass-epoxy laminate | 5 | 20 |

Advantageously, the electrically insulating zone is defined by a relative permittivity of greater than 0.5, preferably greater than 1, and preferably greater than 2.5, when the article is dry.

Advantageously, the electrically insulating zone is defined by an electric strength of greater than 3 kV/mm, preferably greater than 4 kV/mm, and preferably greater than 20 kV/mm.

These values are optimal for obtaining an optimal electrical insulation between the compartments of the article.

Preferably, the electrically insulating zone comprises a material selected from electrically insulating polymers, electrically insulating ceramics or air.

Dimensions

The electrically insulating zone may have a width of between 2 and 10 mm, for example between 2 and 5 mm.

The electrically insulating zone may have a thickness of between 0.2 and 1 mm, and preferably between 0.2 and 0.5 mm.

The electrically insulating zone may be straight and/or curved. Advantageously, the electrically insulating zone may take the form of a straight strip.

Electrically Insulating Polymers

Preferably, the electrically insulating zone comprises a material selected from insulating thermoplastic materials, insulating thermosetting materials, insulating silicones, insulating thermoplastic elastomers, polyester-based or polyether-based insulating thermoplastic polyurethanes or PVC-based insulating thermoplastic elastomers.

The electrically insulating zone advantageously comprises one of the following insulating thermoplastics: polyamides (PA), polyolefins or polyalkenes (for example polyethylene PE, polypropylene PP, polymethylpentene PMP, polybutene PB-1, polyethylene terephthalate PET), styrene polymers (for example, polystyrene PS, expandable polystyrene EPS, acrylonitrile butadiene styrene terpolymer ABS), polyacrylics (polymethyl methacrylate PMMA) or else vinyl polymers (for example polymethyl vinyl ether PMVE, polyvinyl acetate PVAc, polyvinyl chloride PVC).

Alternatively, the electrically insulating zone advantageously comprises the following insulating thermosetting materials: polyurethanes (PU) originating from the reaction of an isocyanate with hydroxylated groups in order to form a flexible open-cell foam suitable for contact with the skin.

Alternatively, inorganic polymers, the main chain of which does not comprise carbon atoms, are advantageously used as constituent material of the electrically insulating zone, especially polysiloxanes or silicones in common parlance. Some examples of the silicones used are polydimethylsiloxane (PDMS), silicone rubber comprising methyl and phenyl groups (PMQ), silicone rubber comprising methyl, phenyl and vinyl groups (PVMQ) or else silicone rubber comprising methyl and vinyl groups (VMQ).

The material constituting the separation zone may also advantageously be selected from insulating thermoplastic elastomers TPE, such as thermoplastic styrene elastomers (for example butadiene and styrene copolymers SBS and ethylene, butylene and styrene copolymers SEBS), thermoplastic polyurethanes TPU based on polyester (AU) or based on polyether (EU), and thermoplastic elastomers based on PVC (TPE/PVC).

The material constituting the electrically insulating zone may also be an ink, such as that described in WO 2009150972, EP-A-0 016 498 or EP 0 168 849.

The material constituting the electrically insulating zone may be deposited on the article by pressurized jet followed by drying and evaporation of the solvents or by the screen printing process. The material constituting the electrically insulating zone may be chemically impregnated on the article.

### Creation of the Separation Zone

The process for creating the electrically insulating zone comprises the steps consisting in:
providing a woven or nonwoven support,
laying the support flat and optionally stretching it in order to have a flat surface,
defining the size and the geometry of the electrically insulating zone,
optionally, marking the electrically insulating zone with a boundary, in particular with a mold,
choosing an electrically insulating polymer, the melting point of which is markedly below the melting point of the constituent material of the support,
depositing the electrically insulating polymer in the molten state at the boundary,
allowing the electrically insulating polymer to cure by cooling down to ambient temperature,
optionally, removing the mold.
optionally, turning the support over and carrying out the same steps again on the reverse side of the support.

Another implementation process is screen printing of the insulating polymer ink on the support.

The steps described above are part of a process of “double printing” of a polymer on a support. This process advantageously makes it possible to have a deep and uniform diffusion of the material of the electrically insulating zone in the support. An electrically insulating barrier is formed between the compartments, preventing the migration of the active components out of the desired area.

The electrically insulating zone, made of one of the electrically insulating materials cited above, adheres perfectly to the body area, in particular to the skin, at its contact surface. This adhesion ensures the absence of moisture or air between the contact surface with the skin. Furthermore, once the active components of the compartments have been deposited at the surface of the skin, these components do not intermingle in the neighbouring deposition area. The active components therefore remain concentrated on the body area for which they are intended. This concentration improves the penetration of the components into the skin via a two-fold mechanism: occlusion and iontophoresis.

### Cosmetic Composition

The first cosmetic composition and the second cosmetic composition generally comprise at least one active principle.

The active principle is, preferably, charged. The term “charged” is understood to mean any active principle present at least partially in ionic form, the ions of which have either a positive or a negative net charge, capable of ensuring their mobility within the composition under the effect of an electric field. Thus, the active agent is directly subjected to the attraction or repulsion of the electrodes.

Advantageously, the first and second cosmetic compositions are selected from care, washing, purifying, exfoliating, desquamating, massage, slimming, makeup, makeup removal, cleansing or bleaching compositions.

More advantageously, the first composition and the second cosmetic composition take the form of an aqueous solution, an oil, an emulsion, a powder or a gel.

The first composition and the second composition may include different respective active principles of opposite charges.

The two compositions preferably take the form of gels.

Irrespective of the embodiments considered, the article may exert an action on the skin via iontophoresis and/or electroosmosis.

The article allows the migration of one composition toward two contiguous regions of a body area to be promoted. The two compositions may be identical or different.

The composition may comprise a composition for activating an inactivated active principle present within the article, for example in freeze-dried form. In this case, the composition may be free of a charged active principle. The composition may comprise a solvent having positively and negatively charged species, for example an ionic aqueous solution or an aqueous solution of deionized water or else a solution of NaCl.

It is also possible for the user to apply an activation composition, for example a solvent, to the article. For example, the user may apply running water, when no water is provided within one and the same packaging with the article.

In order to bring the article and the activation composition into contact, the user may pour the composition onto the article. The latter is, for example, present in a pouch or tray enabling the composition to be poured thereon. As a variant, the user may apply the composition to the skin, then apply the article on top.

Advantageously, the first cosmetic composition and the second cosmetic composition are different from each other. In this case, two body areas of different natures may be treated with the same article.

### Electrochemical Reaction

Generally, when it is sought to administer an active principle using the mask according to the invention, said active principle has the same polarity as the electrode. For example, the compounds containing active principles of positive polarity/charge, such as vitamin A, tocopheryl acetate or other active principles of positive charge/polarity, may be combined with an electrode of positive polarity.

The compounds containing active principles of negative polarity/charge such as retinyl palmitate, tocopherol or mandelic acid may, for their part, be combined with an electrode of negative polarity.

It is possible to treat an area of the body with an article, the compartments of which all enable the administration of active principles of one and the same polarity.

It is also possible that certain compartments enable the administration of an active principle of one polarity and the other compartments allow the administration of an active principle of opposite polarity. In any case, the polarity of the electrodes and of the counter electrodes is determined by the polarity of the active principles of each compartment.

### The Support

### Dimensions and Structure

The support may, in the unfolded state, have a largest dimension of between 15 and 80 cm and for example between 25 and 60 cm.

The support may, in the unfolded state, have a thickness of between 0.1 cm and 1 cm, and preferably between 0.2 cm and 0.5 cm.

The support may be configured in order to produce a sound and/or light signal that makes it possible, for example, to alert the user that it has been left on the skin for the required time.

After use, the article may be thrown away in its entirety.

### Insulating Layer

The support may include an insulating layer, which may allow the first electrode and the second electrode to be fastened together. The insulating layer may be flexible.

The insulating layer may include any electrical insulator, for example chosen from insulating thermoplastics such as polyamides, styrenes, polyolefins or vinyls.

The electrode materials may be fastened to the insulating layer by adhesive bonding, hot rolling, welding or soldering.

Conductive tracks may be formed on the insulator by printing, etching, transfer or screen printing.

### Support Material

Advantageously, the support comprises a nonwoven material. The support is thus more flexible. It adapts better to the contours of the chosen area.

### Nonwoven

Within the meaning of the present invention, the term “nonwoven” is understood to mean a substrate comprising fibers, wherein the individual fibers or the filaments are arranged in a disordered manner in a structure in the form of a sheet. The fibers of the nonwoven are generally bonded together, either under the effect of a mechanical action, or under the effect of a thermal action, or by addition of a binder.

Such a nonwoven is, for example, defined by standard ISO 9092 as a web or sheet of directionally or randomly orientated fibers, bonded by friction and/or cohesion and/or adhesion, excluding paper and products obtained by weaving, knitting or stitching incorporating binding yarns or filaments.

Advantageously, the weight percentage of hot-melt fibers contained in the nonwoven is greater than 0.5% and is less than or equal to 100%, advantageously between 5% and 80%.

The hot-melt fibers are for example polyolefin fibers, such as polyethylene (PE), polypropylene (PP) or polyethylene terephthalate (PET) fibers or acrylic fibers such as polymethyl methacrylate (PMMA) fibers, polyurethane fibers or fibers of the following thermoplastics: polyvinyl chloride (PVC), styrene polymers (for example polystyrene PS, expandable polystyrene EPS, acrylonitrile butadiene styrene terpolymer ABS, styrene acrylonitrile copolymer SAN, styrene butadiene copolymer SB), polyamides (PA), polycarbonates (PC), saturated polyesters (for example polyethylene terephthalate glycol PET, polybutylene terephthalate glycol PBT), polyacetals (for example polyoxymethylene POM, trioxane ethylene oxide copolymer), polyvinyl alcohol (PVA), or else fluoropolymers (for example polytetrafluoroethylene PTFE, polyvinylidene fluoride PVDF, polychlorotrifluoroethylene PCTFE).

The other fibers constituting the nonwoven are for example synthetic fibers derived from petroleum derivatives, natural fibers derived from plants or animals, and/or modified natural fibers, derived for example from treatment or regeneration processes in order to form fibers.

The nonwoven may be formed from one or more consolidated fiber webs.

### Conductivity of a Nonwoven

The conductivity of a nonwoven array depends on the type, the amount, the orientation and the distribution of the incorporated conductive fibers. A nonwoven may have a resistance of less than 1500 ohms/square, or else less than 100 ohms/square, for example less than 10 ohms/square.

The conductivity of a sample is calculated as being the quotient of the resistance of the sample, expressed in ohms, divided by the ratio of the length to the width of the sample. The resulting resistance of the sample is expressed in ohms per square. More specifically, the resistance measurement may be in accordance with the “Method for Determining the Resistivity of a Printed Conductive Material” ASTM F1896-98 as described in patent application WO 2009/144684.

The invention also relates to a cosmetic method comprising the steps consisting in:

installing an article as defined above on a body area, in particular on the face,

making an electric current flow within said article thus installed.

Another subject of the invention is an article for the cosmetic treatment of a body area by an electric current, comprising:

(i) a support including at least two compartments each including one electrode, said compartments being separated by an electrically insulating zone,

(ii) at least one counter electrode.

By dividing the iontophoresis article into a plurality of compartments, it becomes possible to increase the effectiveness of the mask while respecting regulatory currents.

In particular, the article according to the invention is a mask for the face.

Advantageously, the electrically insulating zone is designed to prevent the diffusion of a cosmetic composition between the compartments.

It is a zone that is impermeable to fluids or else a zone that is leaktight with respect to fluids.

### DESCRIPTION OF THE FIGURES

The invention may be better understood from reading the following description of nonlimiting illustrative embodiments thereof and from examining the appended drawing, in which:

FIG. 1 is a schematic front view of an exemplary embodiment of an article according to the invention,

FIG. 2 schematically shows a variant embodiment of the article,

FIG. 3 is a schematic front view of an exemplary embodiment of an article according to the invention,

FIG. 4 shows a cross-sectional view of the exemplary embodiment in FIG. 3,

FIG. 5 shows a cross-sectional view of another exemplary embodiment of the article according to the invention,

FIG. 6 is a graph showing the effectiveness of a mask according to the invention relative to a standard mask.

In the figures, the actual relative proportions of the various elements have not always been respected, for the sake of clarity.

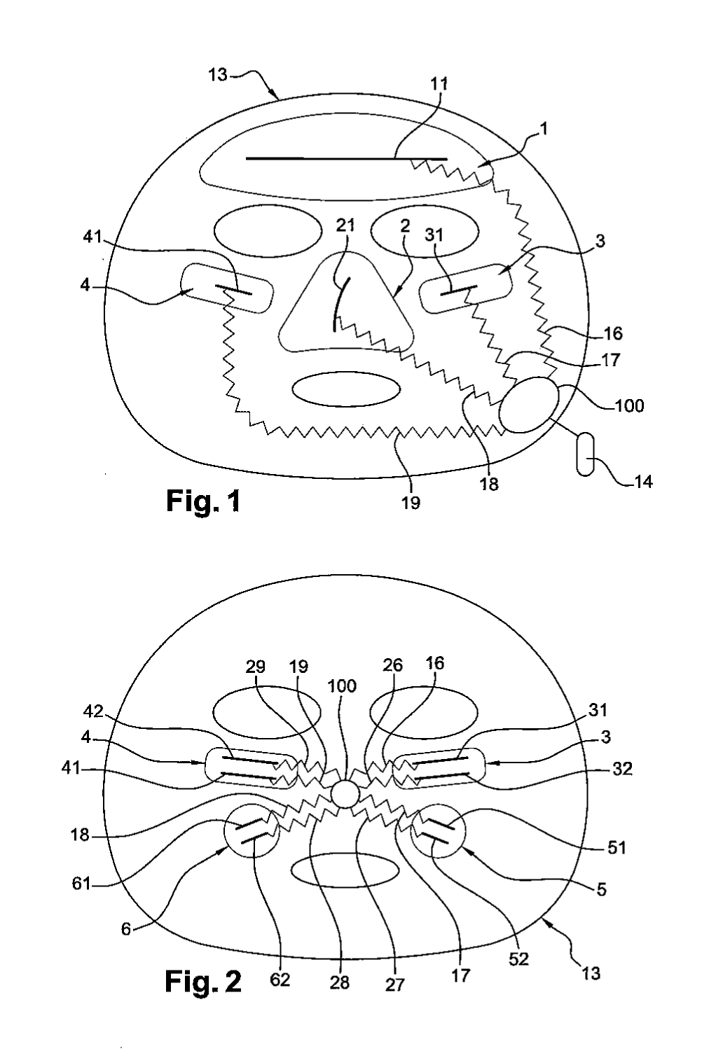

FIG. 1 shows a mask according to the invention configured to cover the face. This mask includes a nonwoven support 13 with four compartments:

a compartment 1 for covering the forehead,

a compartment 2 for covering the nose,

two compartments 3 and 4 for covering the contour of the eyes.

Each compartment comprises an electrode:

the electrode 11 extends over almost the entire length of the compartment 1,

the electrode 21 extends over almost the entire height of the compartment 2,

the electrodes 31 and 41 extend over almost the entire length of the compartments 3 and 4.

In the illustrated example, the mask is configured to treat almost the entirety of the face, i.e. both the contour of the eyes, the nose and the forehead F.

The mask may even, for example, take the form of a balaclava.

The mask may, if appropriate, include means for fastening it to the face.

The mask may, as shown in FIG. 1, contain an electric power source 100 inside it. The electric power source 100 may, for example, be a battery or an accumulator. The electric power source 100 is for example a flat and flexible battery of the Enfucell type.

A housing may be produced within the mask to accommodate the electric power source 100. This housing is, for example, cut into the thickness of one or more layers of the nonwoven.

The electric power source 100 may include:

an electric terminal connected to the conductive tracks 16, 17, 18 and 19,

an electric terminal connected to the counter electrode 14.

When the mask is in place on the face, the counter electrode 14 is held in the hand of the person.

Electric current may be supplied to the electrodes 11, 21, 31, and 41 by way of conductive tracks 16, 17, 18 and 19. The latter may, for example, be made of metal or formed by a conductive ink.

The electrodes 11, 21, 31, 41 may or may not be made of metal. They may, for example, include silicones that have been made conductive, metal wovens or conductive polymers. Where appropriate, each electrode 11, 21, 31 and 41 is integrally formed with its respective conductive track.

The electrical conductivity of the electrodes 11, 21, 31 and 41 may even be related to the presence therein of an electrically conductive electrolyte, a substrate that itself may or may not be electrically conductive possibly being impregnated with this electrically conductive electrolyte.

The exterior surface of the mask may be defined at least partially, or even totally, on the side opposite the skin, by an insulating support, which may extend over a larger area than the electrodes 11, 21, 31 and 41.

The insulating support may allow the various subjacent elements to be fastened together.

This insulating support may be flexible and, for example, selected from insulating thermoplastic films, for example films selected from polyamides, styrenes, polyolefins or vinyls.

The conductive tracks 16, 17, 18 and 19 may be formed by etching the insulating support, or by screen printing or even be applied by transfer. Other processes allowing the conductive tracks 16, 17, 18 and 19 to be added to the insulating support are also possible.

Table 1 below summarizes the active agents delivered by each compartment of the mask in FIG. 1 and the polarity of the electrodes.

TABLE 1

| Compart- | Type of active | Polarity of the | | Polarity of |
|---|---|---|---|---|
| ment | principle | active agent | Electrode | the electrode |
| 1 | “sebum- | retinoic acid: | 11 | negative |
| | regulating” or | negative | | |
| | “anti- | | | |
| | seborrhoeic” | | | |
| | agents | | | |
| 2 | “sebum- | retinoic acid: | 21 | negative |
| | regulating” or | negative | | |
| | “anti- | | | |
| | seborrhoeic” | | | |
| | agents | | | |
| 3 | anti-wrinkle | ascorbic acid: | 31 | negative |
| | agents. | negative | | |
| 4 | anti-wrinkle | ascorbic acid: | 41 | negative |
| | agents. | negative | | |

| | |
|---|---|
| Polarity | Positive |
| of the | |
| counter | |
| electrode 14 | |

FIG. 2 schematically illustrates another exemplary mask according to the invention.

This mask includes a nonwoven support 13 with four compartments:

two compartments 3 and 4 for covering the contour of the eyes,

two compartments 5 and 6 for covering the cheek bones.

Each compartment comprises an electrode 31, 41, 51 and 61.

It may be seen that the counter electrodes 32, 42, 52 and 62 are placed in each compartment 3, 4, 5 and 6 directly facing the electrodes 31, 41, 51 and 61. In this case, the hands of the user are free. They do not hold one or more counter electrodes.

The mask may comprise, within it, an electric power source 100.

The electric power source 100 may include:

an electric terminal connected to the conductive tracks 16, 17, 18 and 19, and

an electric terminal connected to the conductive tracks 26, 27, 28 and 29. Electric current may be supplied to the electrodes 31, 41, 51, and 61 by way of conductive tracks 16, 17, 18 and 19.

Electric current may be supplied to the electrodes 32, 42, 52, and 62 by way of conductive tracks 26, 27, 28 and 29.

An insulating layer may be interposed locally between the skin of the user and the network of electrodes. This insulating layer may allow the comfort of the mask to be increased and the electric power source 100 and the network of electrodes to be retained within the mask. It also allows the power source 100 to be electrically insulated from the skin of the user.

As in the example in FIGS. 1 and 2, the conductive tracks may cover the electrodes only slightly.

The electric tracks may have other shapes than those shown. In particular, they may be branched. It is also possible for the conductive tracks to have a substantially circularly arcuate structure that better follows the contours of the face.

Table 2 below summarizes the active agents delivered by each compartment of the mask in FIG. 2 and the polarity of the electrodes.

TABLE 2

| | | | | Polarity |
|---|---|---|---|---|
| | | | | of the |
| Compart- | Active | Polarity of the | Polarity of | counter |
| ment | principle | active agent | the electrode | electrode |
| 3 | anti-wrinkle | hyaluronic | 31: negative | 32: positive |
| | agents. | acid: | | |
| | | negative | | |
| 4 | anti-wrinkle | hyaluronic | 41: negative | 42: positive |
| | agents | acid: | | |
| | | negative | | |
| 5 | anti-wrinkle | lactic acid: | 51: negative | 52: positive |
| | agents | negative | | |
| 6 | anti-wrinkle | lactic acid: | 61: negative | 62: positive |
| | agents | negative | | |

It is also possible, for example, to use. for the mask in FIG. 2, the compositions and electrodes indicated in table 3. The electric tracks must in this case be arranged so as to be suitably connected to the corresponding polarities of the electric power source 100.

TABLE 3

| | | | | Polarity |
|---|---|---|---|---|
| | | | | of the |
| Compart- | Active | Polarity of the | Polarity of | counter |
| ment | principle | active agent | the electrode | electrode |
| 3 | anti-wrinkle | hyaluronic | 31: negative | 32: positive |
| | agents | acid: | | |
| | | negative | | |
| 4 | anti-wrinkle | hyaluronic | 41: negative | 42: positive |
| | agents | acid: | | |
| | | negative | | |
| 5 | anti-wrinkle | Vitamin A | 51: positive | 52: negative |
| | agents | | | |
| 6 | anti-wrinkle | Vitamin A | 61: positive | 62: negative |
| | agents | | | |

To use the masks shown in FIG. 1 or 2, the procedure is as follows:

The mask is placed on the face.

The user triggers the operation thereof via a control button.

The treatment time varies between 15 and 20 min, the limiting factor possibly being the battery operating time.

Once the treatment is finished, the mask is not reused but it could be reused some other way.

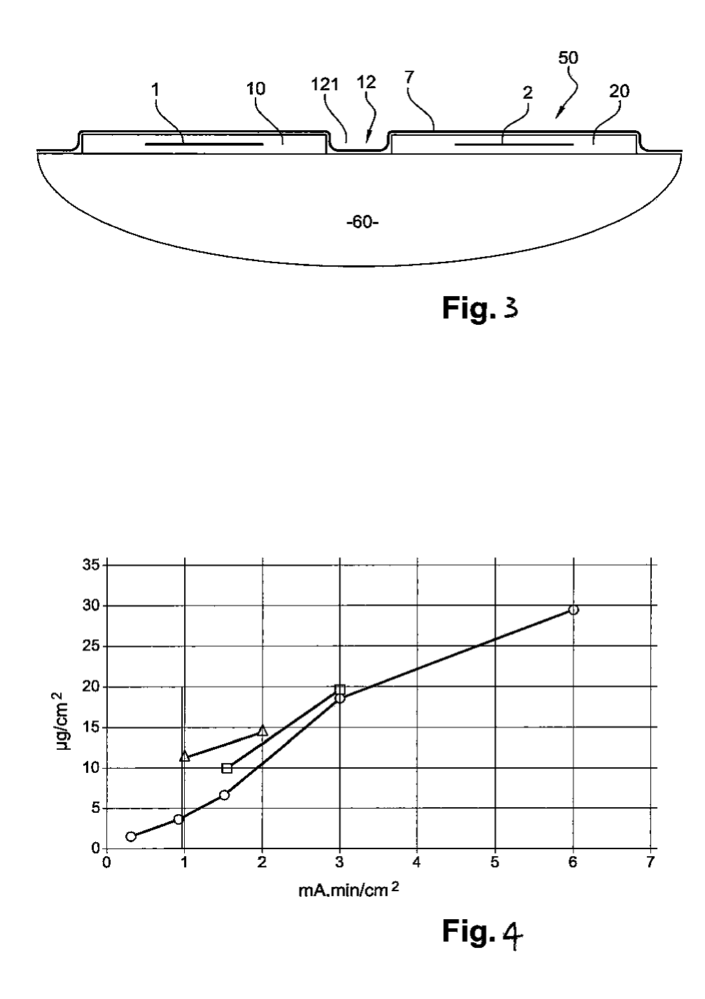

In FIGS. 3 to 6, the actual relative proportions of the various elements have not always been respected, for the sake of clarity.

The article shown in FIGS. 3 to 6 is a mask for the face.

The masking includes a support 50 and a counter electrode 40.

It comprises three compartments 10, 20 and 30.

Each compartment comprises an electrode 1, 2 and 3.

The compartments 10, 20 and 30 are separated from one another by electrically insulating zones 23, 12 and 13.

The article comprises a handle with the counter electrode 40 on the outside thereof and three cables connected to each compartment 10, 20 and 30.

Found in the handle are the current generator, the control buttons, the battery and a programmable switch. The latter makes it possible to power the three cables in a sequential or synchronized manner as necessary. The maximum total current may range up to 5 mA.

In another variant of the mask, the counter electrode 40 is placed in an area right next to the face. Current is thus prevented from passing through the heart and sensitive organs (nerves and muscles). A better focus is obtained on the penetration of the active agents into the skin. Furthermore, the maximum total current is increased above 5 mA without running any risks in terms of electrical safety.

FIG. 4 represents a cross-section of the nonwoven mask from FIG. 1 with two compartments 10 and 20. They are separated by an electrically insulating zone 12. This zone is created by double printing of an insulating barrier ink. Thus, the ink penetrates deeply into the fibers of the support 50. The barrier ink creates an electrically insulating zone. Each compartment 10 and 20 may be impregnated with a cosmetic composition.

In this case, the barrier ink prevents the conductive cosmetic composition from migrating into the thickness of the support 50.

An electrically insulating hermetic film 7 adheres to the support at the barrier ink. It is chosen so that the flexibility of the whole of the mask is preserved. With the barrier ink, it delimits an electrically insulating zone 12 which moreover prevents the cosmetic composition from migrating from one compartment 10 to the other 20 by bypassing the barrier ink.

Furthermore, the electrically insulating zone 12 generates good contact with the skin 60 by adhesion. The conductive cosmetic composition cannot pass from one compartment to the other. A good electrical insulation between the electrodes 1 and 2 is ensured.

This method of manufacturing the mask as multiple layers also ensures a long-lasting moisture of the mask throughout the treatment time. It also improves the penetration performance of the cosmetic active agents through the two-fold mechanism of occlusion and iontophoresis.

The printed barrier-ink track does not need to be very wide, from 2 mm to 5 mm is sufficient to create optimal insulation between the treatment areas.

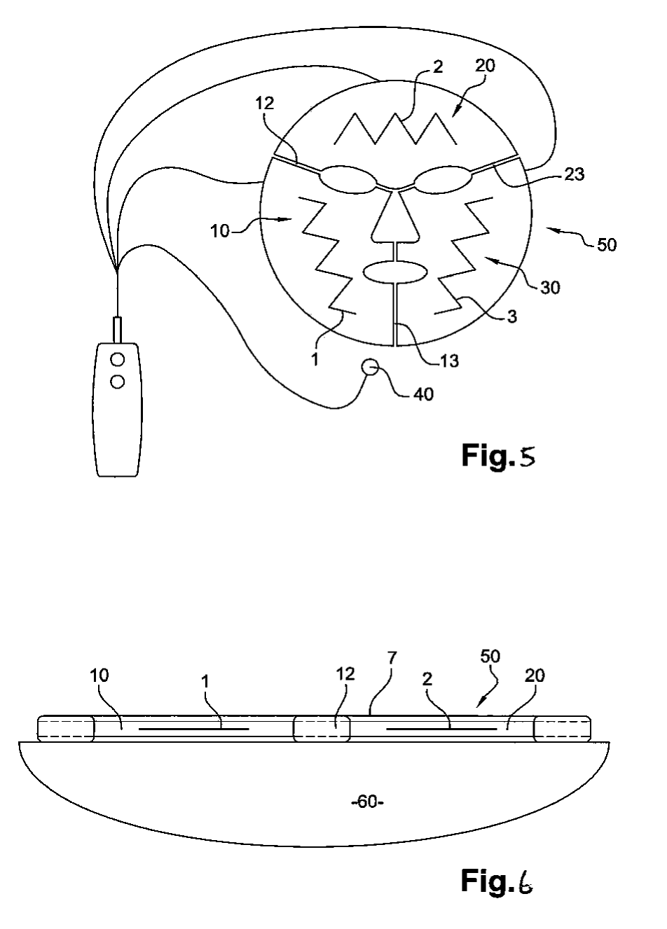

FIG. 5 represents a cross-section of another nonwoven mask with two compartments 10 and 20. They are separated by an electrically insulating zone 12. This zone 12 is formed by:

air 121 and,

an electrically insulating hermetic film 7, for example a flexible adhesive tesa® PVC insulating film.

The hermetic film 7 is treated on its internal surface with an adhesive agent in order to allow it to adhere to the compartments 10 and 20. The support 50 is covered with Teflon® on the surface thereof making contact with the film. The support 50 is also precut into separate compartments 10 and 20. After the support 50 has, for example at the point of use, been impregnated with a chemical composition, the mask is applied to the face. The adhesive treatment on the internal surface of the film ensures an adhesion to the skin 60 and consequently creates the separate and independent treatment areas.

FIG. 6 shows the variation in the amount of the active agent LR2412® that penetrates into the skin as a function of the dose of current delivered by the mask in FIGS. 1 and 2.

In this figure:
the triangles represent the points obtained for the cream Visionnaire® sold by Lancôme with a standard generator for a current equal to 0.1 mA/cm²,
the squares represent the points obtained for the cream Visionnaire® sold by Lancôme with a Hitachi generator at 0.15 mA/cm²,
the circles represent the points obtained for the cream Visionnaire® sold by Lancôme with a standard generator at 0.3 mA/cm².

Comparison of the curves shows that for the same electric dose (mA.min/cm²), the penetration of LR2412® reaches its optimum with a certain intensity of current.

For this mask, the dose for 30 minutes of treatment is 10 mA*30 mins/300 cm²=1 mA.min/cm2.

In this case, the current that gives the best penetrated amount of active agent is 0.1 mA/cm².

This explains why the mask is cut into 3 areas and not into 2 or 4 or 5 areas.

In order to use the mask presented in FIG. 1, the procedure is performed as follows.

The mask is placed on the face.

The user triggers the operation thereof via a control button.

The treatment time varies between 15 minutes and 1 hour, the limiting factor possibly being the battery operating time.

Once the treatment is finished, the mask is not reused but it could be reused some other way.

The invention is not limited to the examples that have just been described.

The implementation characteristics of the examples illustrated may be combined together within variants that are not illustrated. The structure of the electrodes and the surface area that they occupy in each compartment may in particular be different. The nature of the support, and the number and the size of the compartments may be different. The same is true for the nature of the chemical compositions and the polarity of the electrodes.

The expression “comprising a” should be interpreted as being synonymous with “comprising at least one”.

1. An article for the cosmetic treatment of a body area by an electric current, comprising:
(i) a support (13) including:
a first compartment (3, 10) comprising a first cosmetic composition and a first electrode (31, 1),
a second compartment (6, 20) comprising a second cosmetic composition and a second electrode (61, 2),
(ii) at least one counter electrode (14, 32, 62).

2. The article as claimed in claim 1, comprising a first counter electrode (32) and a second counter electrode (62).

3. The article as claimed in any one of the preceding claims, including an electric power source (100) located on the support (13).

4. The article as claimed in any one of the preceding claims, the first cosmetic composition and the second cosmetic composition being different from each other.

5. The article as claimed in any one of the preceding claims, said compartments (10, 20, 3, 6) being separated by an electrically insulating zone (12).

6. The article as claimed in claim 5, the electrically insulating zone (23, 12, 13) being designed to prevent the diffusion of a cosmetic composition between the compartments (10, 20).

7. The article as claimed in either one of claims 5 and 6, the electrically insulating zone (3, 12, 13) having an electrical conductivity of less than 10⁻⁶ S.m⁻¹ and preferably of less than 10⁻¹² S.m¹.

8. The article as claimed in one of claims 5 to 7, the electrically insulating zone (23, 12, 13) being defined by a relative permittivity of greater than 0.5, preferably greater than 1, and preferably greater than 2.5, when the article is dry.

9. The article as claimed in one of claims 5 to 8, the electrically insulating zone (23, 12 or 13) including a material selected from electrically insulating polymers, electrically insulating ceramics or air.

10. The article as claimed in one of the preceding claims, which article is powered by a sequential current the basic element of which is a DC current, an AC current or a pulsed current.

11. The article as claimed in one of the preceding claims, comprising a generator designed so that the user can change the polarity of the current.

12. The article as claimed in any one of the preceding claims, the first and second cosmetic compositions being selected from care, washing, purifying, exfoliating, desquamating, massage, slimming, makeup, makeup removal, cleansing or bleaching compositions.

13. The article as claimed in any one of the preceding claims, the support (13) comprising a nonwoven material.

14. The article as claimed in any one of the preceding claims, the first composition and the second composition taking the form of an aqueous solution, an oil, an emulsion, a powder or a gel.

15. A cosmetic method comprising the steps consisting in:
installing on a body area, in particular on the face, an article as claimed in any one of the preceding claims,
making an electric current flow within said article thus installed.
